(19) United States
(12) Patent Application Publication
(10) Pub. No.: US 2019/0224285 A1
(43) Pub. Date: Jul. 25, 2019

(54) PHARMACEUTICAL COMPOSITION FOR ORAL ADMINISTRATION COMPRISING LACTASE AND PROCESS FOR PRODUCING SAME

(71) Applicant: PRATI, DONADUZZI & CIA LTDA, Toledo (BR)

(72) Inventors: Luiz DONADUZZI, Toledo (BR); Carmen Maria DONADUZZI, Toledo (BR); Liberato BRUM JUNIOR, Toledo (BR); Vanderson GALAN, Toledo (BR); Natália NIEDERMAYER WAGNER, Toledo (BR); Érica Rosane KOVALCZUK, Toledo (BR)

(73) Assignee: PRATI, DONADUZZI & CIA LTDA, Toledo (BR)

(21) Appl. No.: 15/778,461

(22) PCT Filed: Oct. 3, 2017
(86) PCT No.: PCT/BR2017/050300

§ 371 Date: May 23, 2018

(30) Foreign Application Priority Data
Oct. 4, 2016 (BR) 102016023115-9

(51) Int. Cl. A61K 38/47; A61K 9/00; A61K 9/20

(52) U.S. Cl. CPC A61K 38/47; A61K 9/0056; A61K 9/2054; C12Y 302/01108; A61K 9/2018; A61K 9/2009; A61K 9/2027

(57) ABSTRACT

The present invention falls within the fields of nutrition and pharmacy, being related to an orally disintegrating tablet comprising the lactase enzyme in combination with pharmaceutically acceptable excipients, and to a process for preparing same. The tablet of the present invention, as a result of disintegrating in a reduced amount of time, can be orally administered, making it easier to swallow and dispensing with the simultaneous consumption of liquid, and can also be mixed with lactose-rich foods, inducing enzyme activity outside of the organism.

### FIELD OF THE INVENTION

The present invention falls within the fields of nutrition and pharmacy, being related to an orally disintegrating composition comprising an lactase enzyme for use to aid in metabolism of milk derivates.

### BACKGROUND OF THE INVENTION

This report aims to show a bibliographic review and scientific data of lactase use in oral pharmaceutical forms in order to improve the user metabolism of lactase.

The lactase enzyme hydrolyzes lactose, disaccharide found in milk and derivatives. The lactose breaks down into galactose and glucose is one of steps to sugars metabolism that shows failure in subjects who do not endogenously produce the enzyme in sufficient amounts. To aid this break down, lactase allows the glucose and galactose monomers to be absorbed by bowel membrane, and used as energy source by the body.

The lactase use aiming to hydrolyze lactose is allowed in Brazil as a milky drinks co-adjuvant technology (IN MAPA 16/2005). Such use allows the intake of such drinks by people with lactose intolerance; however the ingestion of milk derivatives is restricted to enzyme-treated products during manufacture.

The purpose of product use is to assist in lactose break down originated from milky foods, in order to facilitate the digestion of such foods. In compliance with ANVISA guidelines [National Health Surveillance Agency] included in Technical Report #65, Feb. 23, 2015, clarifying about the enzyme use in foods and drinks, it is required the registration of lactase-based product representing functional propriety.

### Scientific Evidences Applicable to Safety Proof

The safety of lactase for human consumption is already well established and has an extensive use history, since it is used for decades for producing foods and also as supplement. Its characteristics are described in many publications and text books.

Exogenous lactase shows strictly the local of action (in small bowel) and its systemic absorption is irrelevant. Thus, there is no registration in literature about studies on exogenous lactase dosing in blood. Biodisponibility data, area under curve (AUC) and maximum concentration (Cmax) are not applicable. There is no description of exogenous lactase pharmacokinetic profile however given its exclusively local of action in intestinal lumen, it is supposed that it is cleaved and absorbed in amino acids form, like any other protein, and the removal of non-digested portion occurs directly through feces.

In report of 15th JECFA Meeting (Joint Expert Committee of Food Additives), the committee appointed the risk involved the consumption of food-incorporate enzymes when they originate from microorganisms, mainly due to the risk of mycotoxins presence. However, it was concluded that, even inhalation as well as skin contact with enzymes may induce in allergic reactions, when enzymes are consumed, they do not induce more allergies than any other food ingredients.

Thus, it is evident that the safety of using enzymes is directly related to strict control of Good Manufacturing Practices, in order to avoid contamination, and to culture control used in enzyme production.

Proof of Traditional Use, Seen in Population, without Association to Health Damages.

As mentioned previously, lactase is an enzyme naturally present in human's bowel. Lactase obtained from microorganism cultures, including A. oryzae, is used in food industry as co-adjuvant of manufacturing technology, being approved by RDC 53/2014, define authorized enzymes for food use, generally intended to human consume. Therefore, such enzyme has already been assessed by Agency regarding its safety use.

The addition of lactase enzyme in food products, aiming to decrease the lactose concentration, is a practice adopted for a long time in Brazil and abroad. In all this foods, lactase enzyme is not removed from “treated” food and it is consumed by consumer along with final product. Therefore, it can be assumed that enzyme consumption as food ingredient already is a common practice by population, which uses products having decreased lactose content through the addition of lactase enzyme.

Lactase in tablet forms is sold in several countries, such as: The United States, United Kingdom, Canada, Mexico, Japan, The Switzerland, Italy, Turkey and Australia. In all mentioned countries, the product is considered a food supplement and there are no reported cases of adverse events.

At website of Food and Drug Administration (FDA), in 2015, that inspects lactase-containing products and lactase-based supplements in the United States, there are no recalls or warnings registrations related to such products.

### Scientific Evidences Applicable to Assertion Proof of Intended Functional Propriety

Lactose intolerance affects about 70% worldwide population. Prevalence varies according to population group ethnicity and it is related to the use of milky products, resulting in genetic selection of subjects capable to consume lactose. For instance, the prevalence is about 80% in black people, almost 100% in Asian people, and only 2% in populations from Nordic Countries in Europe (Swagerty et al., 2002). In Brazil, it is supposed that about 45% of population shows lactose intolerance (Pereira Filho & Furlan, 2004).

To reduce unpleasant effects arising lactose intake, the first action is to delete milk-originated foods from eating habit, what can lead to calcium deficiency and resulting bone weakening in long term. Among alternative strategies recommended by scientific community are the consumption of foods treated previously with lactase, and also supplementation of active lactase, since both of them allows people presenting lactose intolerance consumes milky products.

The efficacy of lactase as supplement in tablets, pills, capsules, and powders, forms which may be used along with meals containing lactose and without need of previous incubation of foods, have already been proven by several scientific studies and literature reviews (Rao, 1997). Consequently, international agencies, like Canadian Health Agency (Health Canada) and EFSA (European Food Safety Authority), have assessed such studies and have recognized the efficacy of active lactase-based products.

EFSA has considered that there are sufficient scientific evidences to proof cause-effect relationship between lactase enzyme consumption as food ingredient and lactose break in subjects showing symptomatic lactose intolerance.

In Brazil, lactase of Aspergillus oryzae expressed in Aspergillus niger is approved by RDC Resolution 53, of Oct. 7, 2014, that establishes the allowable enzyme list as food manufacturing technology co-adjuvant. Recently, it was approved by ANVISA the registration of tablets of Lactase by company Eurofarma Laboratórios S.A.

The lactase use as ingredient to produce supplements in form of capsules, sachets, tablets and other is approved in several countries, including in Brazil, which recently granted registration to product “lactase in tablets”.

The fact of lactase enzyme improves lactose digestion in subjects having difficult to digest lactose is contained in the list of health statements in foods allowed by European Committee (Regulation CE 432/2012).

In the United States, lactase is on the list of Enzyme Technical Association related to enzymes already sold in food supplement form before 1994.

In Canada, lactase as active ingredient in supplements is classified as a Natural Health Product, the same category in which are included foods added by vitamins and/or minerals, amino acids, essential fatty acids and probiotic microorganisms, including juices and waters with vitamins, energy drinks, yogurts and bars. Lactase monograph created by Canadian Health Agency does not present any use restriction or contraindications different than those related to allergy development likelihood, when the use should be discontinued. It is recommended the consumption up to 18000 FCC of lactase per day as digestive enzyme to aid in digesting lactose-containing foods. In order to be approved by Canadian Health Agency, the products are subject to a thorough scientific-technical assessment and they should proof the use safety and efficacy, in compliance with the guidelines and specific laws for Natural Health Products. In addition, Agency verifies whether manufacturing is suited (without contaminations or incorrect ingredients) and monitors post-sell products, in order to ensure that the laws are being followed.

### Clinical Studies

The following presents clinical studies, released in indexed scientific magazines, proofing the efficacy of lactase-based products, in the same situations and/or in the proposed use mode for product in form of orally disintegrating tablets. Some of such studies prove that the consumed lactase in meal times is effective in lactose break down, indicating that the enzyme is not activated in stomach and continues its action in bowel.

Studies Performed with Enzyme Originated from Aspergillus oryzae

A recently published study (Vrese et al., 2015) has assessed lactase efficacy in capsules in hydrolyzing lactose, when consumed immediately before lactose-containing food. This is a randomized, controlled, double-blind and crossover clinical study. Volunteers (n=24) with average age of 31 years and different degrees of lactase deficiency received capsules containing 3300 or 9000 units FCC of lactase de A. oryzae or capsules of placebo, followed by a glass of milk (150 mL) containing about 12.5 g of lactose. The experiments were performed on separate days, with interval of 2 weeks between each capsule type. Exhaled hydrogen was significantly lower after lactase capsule consumption in comparison to placebo, being both (3300 and 9000 FCC) effective amounts. Further, there was decrease in frequency and duration of abdominal pain and flatulence caused by lactose intake.

This study demonstrates efficacy of lactase de A. oryzae, consumed as supplement (capsule) concomitantly to lactose source food, in amounts similar or even lower than that proposed in recommendation of orally disintegrating tablet of Lactase product.

A double-blind, placebo-controlled, crossover clinical study n=134 was performed with main objective to test efficacy of oral intake of tilactase, a β-D-Galactosidase obtained from Aspergillus oryzae, regarding gastrointestinal discomfort and hydrogen excretion in subjects presenting or malabsorption or lactose intolerance (Portincasa et al., 2008). Still, its secondary objective to test de enzyme use safety. The study participants were divided randomly in 3 groups. A group of 72 subjects received lactase tablets at dose of 4600 units FCC; the second group, with 43 people, received a dose of 7700 units FCC and the third group, with 19 people, received two tested doses (in different times). Therefore, 91 people received 4600 FCC and 62 people received 7700 FCC of lactase. Both tested doses and placebo were administrated immediately before drinking 500 mL of milk (approx. 25 g of lactose). Following administration of doses, samples from exhaled air were drawn at every 30 minutes of time 0 up to 240 min and at time of 360 min. Expiration tests for every scenario were performed with interval of 72 hours. Possible adverse effects were monitored during the experiment and 24 hours later, no effect was reported. The results demonstrate that two tested doses were effective, leading to a significant decrease in hydrogen levels and abdominal discomfort (gases and pain) against placebo, both in subjects presenting malabsorption and those intolerants. Study brings dosing expression of lactase in Extract Chemie (EC), wherein 1 unit of FCC=1.46 unit EC. Thus, tested doses represent 4.623 units FCC and 7.705 units FCC.

Thus, this study demonstrates the efficacy of lactase A. oryzae, consumed as supplement immediately before lactose-source food, in amounts lower those proposed in consumption recommendation of present orally disintegrating tablet of lactase product.

In other controlled, crossover clinical study, 10 adult volunteers (average age of 43.5 years old) participated in a trial where they received a solution containing 50 g of lactose in 4 different days, to evaluate the effect of exogenous lactase enzyme consumption in level of exhaled hydrogen and in symptoms of lactose malabsorption subjects. The evaluated product was a powered lactase supplement produced from A. oryzae (tradename Lactase®, manufactured by Kremers-Urban Company, Milwaukee, USA), consumed immediately before lactose solution. In two first days, group received placebo, at third day 250 mg of lactase (4000 units FCC), and at fourth day 500 mg (8000 units FCC) dissolved in water immediately before lactose intake. According to results, the intake of 8000 units FCC of lactase resulted in a significant decrease in exhaled hydrogen in comparison with placebo intake. While it is not significant statistically, the intake of 4000 units of FCC results in a decrease in exhaled hydrogen when compared to placebo intake, thus demonstrating a exhaled hydrogen dose-dependent decrease. Regarding to swelling and flatulence, the two doses entailed a significant decrease (DiPalma & Collins, 1989).

This study demonstrates the efficacy of powered A. oryzae lactase, consumed immediately before lactose-containing food, in dose similar or lower than orally disintegrating tablet lactase product.

Other controlled, crossover clinical study was performed to assess the efficacy of A. oryzae lactase in tablets form (tradename Lactrase®, manufactured by Kremers-Urban Company, Milwaukee, USA). On first visit, 16 volunteers (13-81 years old) with lactose intolerance in fasting consumed 360 mL of milk containing 17 g of lactose (baseline). On the second visit, volunteers consumed 250 mg of lactase (4000 units FCC) along with the same amount of milk. Exhaled hydrogen was significantly lower than baseline in 5 of 16 volunteers. 11 patients who failed in decreasing with such lactase amount (4000 FCC), 7 participated in a new trial in which they received 500 mg of tablet enzyme (8000 units FCC), showing indices of exhaled hydrogen significantly lower those measured with only milk intake (baseline). Authors have concluded that lactase supplementation was effective to break down milk-originated lactose and that it allows consumption of foods that was previously avoided by people who are lactose intolerant, due to its known induction to unpleasant effects arising from lactose content (Moskovitz et al., 1987).

This study also demonstrates lactase efficacy in tablets form, consumed concomitantly with lactose-source food, and at similar dose or lower than orally disintegrating tablet lactase product.

The randomized, placebo-controlled clinical trial by Ramirez et al. (1994) assessed the efficacy of 3 tablets products lactase-based (9900 units FCC), being one of them originated from A. oryzae (product Lactrase®), concomitantly consumed with an ice cream containing 18 g de lactose. In 10 volunteers, all lactase supplements were effective in decreasing exhaled hydrogen and lactase malabsorption-originated symptoms comparing to ice cream consumption+placebo.

Besides to support the conclusion of other studies on exogenous lactase efficacy, this study shows efficacy when consumed other milky matrices other than milk.

Lin et al. (1993) compared the efficacy of three lactase-base products available in the market at the moment of the study: Lactogest® in capsules (Thompson Medical, New York, USA); Lactaid® capsules (Lactaid Inc., New Jersey, USA) and Dairy-Ease® chewable tablets (Gleenbrook Lab, USA). Study does not specify microbial sources of lactase in each product, but in accordance with review by Rao et al. (1997), the products are from three different sources: A. oryzae, K. lactis and A. niger, respectively.

Two randomized, double-blind, crossover, placebo-controlled trials were performed separately, one of them at Minnesota University and the other at Alabama University. Twenty volunteers (age varying from 25 and 40 years old) participated in study at Minnesota, and 11 at Alabama (age varying from 18-60 years old), all of them healthy and diagnosed with lactose malabsorption. Every volunteer was subjected to 5 interventions randomly: Placebo, 2 Lactogest® capsules (2918 units FCC), 4 Lactogest® capsules (5836 units FCC), 2 Dairy-Ease® tablets (5790 units FCC) and 2 Lactaid® capsules (5466 units FCC), concomitantly consumed with 400 mL of milk added with 20 g of lactose or a 200 mL of lactose solution (50 g) in water. In Minnesota's study, three products were effective in peak decreasing and total production of exhaled hydrogen in doses near 6000 FCC, and the arising of colic, nausea, swelling, diarrhea and flatulence was also significantly lower with lactase consumption. In Alabama's study, there was no significant difference, and authors have concluded that consumed amount of lactose (50 g) was superior than the hydrolyze capacity of administered lactase amounts.

This study demonstrates the efficacy of lactase A. oryzae when consumed in supplement form concomitantly with lactose food and the equivalency of lactase activity in different microbial sources, when administered in similar amounts.

The A. oryzae lactase consumed efficacy in active form was also assessed in volunteers of other ages, like children and teenagers, and they are shown as follows.

In a clinical study performed with preschool children (Barillas & Solomons, 1987), two lactase-based products were tested, one in liquid form (Lactaid®, originated from K. lactis), and other powered (Takamine, originated from A. oryzae). Several trials were performed, aiming to check the effective minimal amounts of two lactase preparations to in vivo lactose hydrolyze, i.e., with consumed enzymes in active form, in comparison with the efficacy of the enzyme with pre-hydrolyzed milk. Firstly, intact milk (no hydrolyzed) test was performed containing 12 g of lactose, in order to verify what children showed lactose malabsorption. In a second time, the subjects consumed lactase incubated milk for 24 h, in which total lactase hydrolysis was confirmed in laboratory. Thus, the dose-response trials continued, adding lactase to milk immediately before the consumption. The excess of exhaled hydrogen was eliminated containing 4200 units FCC of lactase originated from Kluyveromyces lactis in the first trial (n=7) and 6635 units FCC of lactase originated from A. oryzae in the second trial (n=9), and higher tested doses were equally effective. The milk-added lactase immediately before consumption efficacy was similar to previously enzyme-hydrolyzed milk efficacy, since the results of exhaled hydrogen along with active enzyme consumption, from both sources, were statistically similar with those results obtained with previously enzyme-incubated milk.

Based on results, a randomized, blind and controlled clinical study with 48 children was performed, being 27 of them presenting lactose malabsorption, in this time, with “optimal” enzymes found in previous trial doses. Children have participated in four trials: intact milk, lactase pre-hydrolyzed milk, milk+lactase K. lactis, and milk+lactase A. oryzae. The exhaled hydrogen results obtained from the lactose malabsorption children group were similar with pre-hydrolyzed milk consumption and with milk+lactase A. oryzae consumption, confirming the efficacy of immediately before consumption milk diluted enzyme. Even in no lactose malabsorption children group (n=21), the pre-hydrolyzed milk consumption and the milk+lactase A. oryzae consumption have decreased exhaled hydrogen, comparing to intact milk. This study proofs that A. oryzae enzyme was effective when consumed concomitantly with milk, and well tolerated by children.

In other study, 16 children and teenagers (3 to 16 years old, average of 10.4 years old) diagnosed with lactose malabsorption received milk with placebo or A. oryzae lactase capsules (375 mg). From these volunteers, only two of them did not present lactose malabsorption symptoms (swelling, gases, abdominal pains). Samples of exhaled hydrogen were drawn every 30 minutes for 5 hours following consumption. There was a significant reduction in maximum hydrogen exhalation following lactase milk consumption comparing with placebo milk consumption. In 14 volunteers presenting malabsorption symptoms, there was significant decrease in clinical symptom score (Biller et al., 1987).

Again, the study proofs that A. oryzae enzyme was effective in aiding lactose digestion when consumed in supplement form, and its use suite for children and teenagers.

In a randomized, double-blind, crossover, and placebo-controlled trial, 18 children (average age 11 years old) with lactose intolerance consumed A. oryzae lactase tablets or placebo, immediately before consumption of a lactose-containing drink (2 g/kg of body weight). Every volunteer ingested 1 tablet containing 3000 units FCC of lactase for every 5 g of ingested lactose. Samples of hydrogen exhaled by respiration were drawn in intervals of 30 minutes just follow the consumption, for two hours. In addition, clinical symptoms were also monitored. Following placebo administration, hydrogen production was 60 ppm, which was associated with abdominal pain (89%), swelling (83%), diarrhea (61%) and flatulence (44%). When lactase was administrated,the hydrogen maximum production was 7 ppm, and clinical symptoms related to intolerance were decreased. The results indicated that lactase enzyme consumption decreases significantly the hydrogen exhalation and the rising of lactose consumption-originated symptoms (Medow et al., 1990).

Therefore, this study confirms the efficacy of lactase in tablets, consumed concomitantly to lactase-containing foods.

In a double-blind, crossover study, 24 volunteers received an only dose of lactase (9900 units FCC) in chewable tablet form or placebo, followed by a cup of milk containing 50 g of lactose. The study informs lactase used is Dairy-Ease® product manufactured by Sterling Health (New York, USA), however it is not clear which is the enzyme-producing microorganism. Exhaled hydrogen (measure used to diagnose lactose intolerance) was measured every 30 minutes for five hours after consumption, in order to plot a graph. With enzyme consumption, the area under the curve of hydrogen was significantly lower than with placebo consumption for 21 subjects, which proofs that there was a significant break down of lactose. The occurrence and severity of abdominal cramps, flatulence and diarrhea were also lower with lactase consumption (Sanders et al., 1992).

This study proofs lactase efficacy chewable tablets, use mode similar those proposed for orally disintegrating tablet lactase product.

### Observation Regarding Lactase Amounts

Some studies (Moskovitz et al. 1987; and Di Palma & Collins 1989, Barillas & Solomons, 1987; Biller et al., 1987) do not show enzyme amounts in units FCC, only in grams or IU. The conversion to FCC units showed in these technical reports was extracted from document issued by EFSA, which performed the conversion at the studies' assessment moment.

It is worth to emphasize that none of studies reported any adverse effect coming from consumption of supplements or lactase-added milky products, which confirms the safety of enzyme use.

The showed studies demonstrated the active lactase efficacy for aiding in lactose digestion, in different consumption situations, being added to milk immediately before consumption or concomitantly ingested with lactose-containing food, including in the solid foods (ice cream) presence.

It is noted that the most studies have assessed the lactase consumption efficacy using milk as food test (sometimes it was added lactose to) or even pure lactose instead of other milky foods, and in many cases the tested lactose amounts were higher than those naturally found in milky foods, as it may be seen in table 1, presenting lactose amounts in several milky products:

TABLE 1
Lactose amount in milky foods

| | Lactose (g) | Lactose (g) in usual |
|---|---|---|
| Food | in 100 g | portion (RDC 359/2003) |
| Whole milk (3% fat) | 5.05 g | 10.1 g/200 mL |
| Skim milk | 5.09 g | 10.2 g/200 mL |
| Milk jam | 4.92 g | 0.9 g/20 g |
| Vanilla ice cream | 4.43 g | 2.6 g/60 g |
| Cottage cheese | 3.87 g | 1.9 g/50 g |
| Greek yoghurt | 2.72 g | 4.1 g/150 g |
| Fresh cheese | 2.32 g | 0.7 g/30 g |
| White cheese | 1.60 g | 0.5 g/30 g |
| Mozzarella cheese | 1.45 g | 0.4 g/30 g |

Source: USDA Table - http://ndb.nal.usda.gov/ndb/foods

Supporting this understanding, Sanders et al. (1992) mentions that amounts of lactose consumption lower than used in his experiment (50 g of lactose), like found in a glass of milk, cheese or pizza portion, ice cream ball, would be likely associated to a lower incidence and severity of lactose consumption-linked discomfort. In addition, he mentions that consumption along with solid foods and “full stomach” would provide a longer time to mix the foods with enzyme, and consequently higher lactose break down.

What be inferred from literature, there are no documents suggesting or anticipating the teachings of the present invention, so that the solution proposed here has novelty and inventive activity outside the state of the art.

Therefore, it may be seen that the marjority of presented scientific studies supported the active lactase consumption efficacy over the hydrolyzing higher amounts of naturally found lactose in meals containing milky foods. Accordingly, it is possible to conclude that consumption of lactase supplements along with other food matrices containing lower amounts of lactose is equally or even more effective, since the lactase activity measure is directly dependent to lactose (substrate) amount.

### SUMMARY OF THE INVENTION

In a first aspect, the invention provides a stable formulation of lactase enzyme for oral use in human patients like orally disintegrating tablet, which may be administered without using water or other liquids, aiding swallowing.

In a second aspect, the orally disintegrating tablet of the present invention uses one or more disintegrating agents, diluents, lubricants, flavourings, sweeteners agents and a diluent.

In a preferential aspect, the disintegration total time of present tablet is lower than 1 minute.

In another preferential aspect, friability index of final product should be lower than 1.5%.

In an optional aspect, the tablet may be used as technology adjuvant, and it may be dispersed on milk or on milky foods with purpose to inactivate the lactose present in food.

In a third aspect, the orally disintegrating tablet of the present invention has a producing process defined according to the following steps: Normalization of materials, mixture of materials, compressing step in pharmaceutical rotating compressor.

The proposed formulations should provide a suitable process ability, with a robust final formulation.

These and other objects of the invention will be immediately appreciated by the well versed in the art, and for companies with interests in the product segment and will be described in sufficient detail to be reproduced in the following description.

### DETAILED DESCRIPTION OF THE INVENTION

This description aims to investigate the inventive concept details, providing examples to facilitate cognition/understanding of the same and providing precise technical data about some ways to achieve the inventive concept of the invention. The detailed description also aims to avoid the third parties repetition, extensive experimentation, financial, time and intellectual activity expenses that inventors/filling performed to solve the technical problems which are settled herein.

In order to avoid doubts regarding interpretation, any feature described in an aspect of the present invention may be used in other aspect of the invention. The word “comprising” means “including”, but not necessarily “consisting in” or “composed by”. In other words, the steps or options listed should not be exhaustive. It is noted that the examples provided in the following description aim to clarify the invention, and should not be understood per se as limiting the scope of the invention.

This project is related to an orally disintegrating composition comprising lactase, and a process for preparing the same.

In a first aspect, the present invention defines a pharmaceutical composition for oral administration being an orally disintegrating tablet comprising the lactase enzyme in combination with pharmaceutically acceptable excipients.

In a concretization of pharmaceutical composition, the pharmaceutically acceptable excipients comprise at least a disintegrating, a lubricant, a flavoring, a sweetener and a diluent.

In a concretization of pharmaceutical composition, disintegrating agent is chosen among crospovidone, croscarmellose, glycated starch, polacrilin potassium, hydroxypropyl cellulose, microcrystalline cellulose or combinations thereof.

In a concretization of pharmaceutical composition, lubricant is chosen among magnesium stearate, sodium stearyl fumarate, stearic acid, sodium citrate or combinations thereof.

In a concretization of pharmaceutical composition, diluent is chosen among several polyols (e.g. maltitol, mannitol and xylitol), microcrystalline cellulose, starch, talc, maltodextrin, sucrose and dextrose or combinations thereof.

In a concretization, the pharmaceutical composition shows total disintegration in up to 1 minute and friability lower than 1.5%.

In a concretization, the pharmaceutical composition is mixed in lactose-rich foods.

In a second aspect, the present invention defines a process for producing the said pharmaceutical composition comprising the step of lactase mixturing with blend of diluents, disintegrating agents, and sweeteners, and next step of compression.

Lactase enzyme, or β-D-Galactosidase, is a tetramer composed by A and B chains pursuant to family 1 of glucohydrolazes and it is in brush-border of mammal small bowel. Its local action occurs through its catalyst activity on hydrolyzes of lactose disaccharide β-1,4 bond exclusively found in milk and milky products, having as products D-galactose and free glucose. This hydrolysis allows glucose and galactose monomers be absorbed by bowel membrane and in such way they may be used by body as energy source. Thus, lactase is an enzyme aiding lactose digestion present in milk-derived foods.

Lactase is also produced by plants, yeasts, bacteria and fungus. Several microbial sources may be used aiming to synthesize lactose. Industrially, obtained lactase is usually originated from fungus (Aspergillus oryzae, Aspergillus flavus and Aspergillus niger), bacteria (Escherichia coli and Lactobacillus bulgaricus) or yeasts (Saccharomyces fragilis, Torula cremoris and Torula utilis).

Lactose is a disaccharide formed by one glucose and one galactose molecule, exclusively found in mammal's milk. Lactase is the responsible enzyme for hydrolyzing lactose, allowing its absorption in monosaccharide form. Children usually have high lactase concentrations. However, after weaning, in many cases occurs a genetically programmed decrease in lactase synthesis, resulting in enzyme activity loss in adults. In these cases, disaccharide remains in intestinal lumen and is fermented by microflora, resulting in discomfort, pain, swelling abdominal, flatulence and diarrhea. Such situation is called primary lactose intolerance, or even “non-persistent lactase”.

The present product is presented in orally disintegrating tablet form, providing between 5000 and 15000 units FCC: 1 unit FCC is defined as the enzyme amount releasing o-nitrophenol compound at rate of 1 mol/min of lactase enzyme in unit portion, under conditions set forth by Food Chemicals Codex (FCC). It is recommended consume 1 tablet before the milk-originated products consumption or according to physician or nutritionist guidelines.

The product is presented in orally disintegrating tablet form so there is no need to drink water, facilitating the product use by the consumer, becoming this presentation novel for the product.

The amounts may vary among different subjects, and they may be adjusted depending on amount of consumed milk derivatives in each meal or according to physician or nutritionist guideline.

Regarding to safety of lactase consumption, it should be attention to the fact that the same contains enzyme-intrinsic proprieties, composed by natural and digestible amino acids, like any other naturally found protein in foods.

Regarding the producer microorganism, Aspergillus niger is used for commercially producing of several enzymes employed in foods and its safety is also well established, which is also the case of donor specimen of gene encoding lactase production, Aspergillus oryzae.

Therefore, the present invention is composed mainly by lactase enzyme in combination with adequate excipients, such as emulsifiers, lubricants, sweeteners, and a diluent.

Disintegrating agents which may be used in the present invention are crospovidone, croscarmellose, glycated starch, polacrilin potassium, hydroxypropyl cellulose (HPC) and microcrystalline cellulose. These excipients may be present in amount from 70.70 to 106.26 mg, corresponding to 14.13% and 21.25% by weight of final tablet, respectively.

Lubricants that may be used in the present invention are magnesium stearate, sodium stearyl fumarate, stearic acid and sodium citrate. These excipients may be present in concentration from 4.25 to 5.75 mg, corresponding to 0.85% up to 1.15% by weight of final tablet, respectively.

There are several flavoring agents that may be used in the present invention and they are divided between flavourings and sweeteners, which are included in formulation to promote a pleasant taste during oral disintegration. They are vanilla taste, chocolate taste, strawberry taste, grape taste, caramel taste, orange taste, lemon taste, apricot taste, cherry taste, mint taste, peppermint taste, apple taste, banana taste, sucralose, acessulfame, stevia, aspartame, glucose, several polyols, agave, cyclamate and saccharine. These excipients may be present in amount from 1.53 to 2.07 mg, corresponding to 0.306 up to 0.414% by weight of final tablet, respectively, for flavourings; and 0.17 to 0.23 mg, corresponding to 0.034% up to 0.046% by weight of final tablet, respectively, for sweeteners.

Diluents that may be used in the present invention are several polyols (e.g. maltitol, mannitol and xylitol), microcrystalline cellulose, starch, talc, maltodextrin, sucrose and dextrose. These excipients may be present in amount from 193.33 to 261.56 mg, corresponding to 38.6% up to 52.32% by weight of final tablet, respectively.

Friability of tablets should be lower than 1.0%, i.e., the mass loss of each tablet when subjected to mechanical tension should not be equal or lower than 5 mg.

As the object of the invention is an orally disintegrating tablet, the disintegrating time should be equal or lower than 1 minute.

Table 2 below summarizes all components of the present invention and its optimal amounts.

TABLE 2
ORALLY DISINTEGRATING TABLETS

| | | CONCENTRATION | |
|---|---|---|---|
| FUNCTION | COMPONENTS | AMOUNT/VOLUME | PERCENTAGE |
| Enzyme | lactase from 10.000 to 500.000 | 76.50 to 103.50 mg | 15.3% to 20.7% |
| | ALU**/g | | |
| Disintegrating | crospovidone, croscarmellose, | 70.70 to 106.26 mg | 14.13% to 21.25% |
| agents | glycated starch, polacrilin | | |
| | potassium, hydroxypropyl | | |
| | cellulose (HPC), | | |
| | microcrystalline cellulose. | | |
| Lubricants | Magnesium stearate, sodium | 4.25 to 5.75 mg | 0.85% to 1.15% |
| | stearyl fumarate, stearic acid, | | |
| | sodium citrate | | |
| Flavourings | Vanilla taste, chocolate taste, | 1.53 to 2.07 mg | 0.306% to 0.414% |
| | strawberry taste, grape taste, | | |
| | caramel taste, orange taste, | | |
| | lemon taste, apricot taste, cherry | | |
| | taste, mint taste, peppermint | | |
| | taste, apple taste, banana taste | | |
| Sweeteners | Sucralose, acesulfame, stevia, | 0.17 to 0.23 mg | 0.034% to 0.046% |
| | aspartame, glucose, several | | |
| | polyols, agave, cyclamate, | | |
| | saccharine | | |
| Diluents | several polyols (mannitol, | 193.33 to 261.56 mg | 38.6% to 52.32% |
| | xylitol, sorbitol, eritritol, | | |
| | isomalt, lactitol, maltitol), | | |
| | microcrystalline cellulose, | | |
| | starch, talc, maltodextrin, | | |
| | sucrose and dextrose | | |

The producing process uses usual equipment for preparing solid pharmaceutical forms, for example:

Vibrating sieves, pharmaceutical mixers (“V” Mixer, Doble-Cone Mixer, BIN Mixer and/or Turbula® Mixer shaker mixer) and rotating-type pharmaceutical compressor.

The invention is illustrated in more details by the following examples.

### EXAMPLES

### Preferential Realizations

The examples shown here are intended only illustrate one of several ways to implement this invention, however, without limiting the scope thereof.

### Example 1

In order to prepare orally disintegrating tablets, according to Example 1, lactase (9,000 FCC) was mixed with polyols mix, for 10 minutes in “V” mixer. Sodium stearyl fumarate was added to previously prepared mixture and mixed for 2 minutes. The ready-to-compress mixture was compressed in tablets employing different compaction presses in a pharmaceutical rotating compressor.

TABLE 3

| # | MATERIAL | Concentration | formulation % |
|---|---|---|---|
| 1 | LACTASE | Eq. a 9,000 FCC | 18.00 |
| 2 | POLYOLS MIX (Maltitol, | 400.00 mg | 80.00 |
| | xylitol and mannitol) | | |
| 3 | SODIUM STEARYL | | |
| | FUMARATE | 10.00 mg | 2.00 |

### Example 2

In preparation, according to Example 2, lactase (9,000 FCC) was mixed with mannitol, microcrystalline cellulose, and flavoring for 15 minutes in “V” mixer. Sodium stearic acid was added in sequence and mixed for 5 minutes. The ready-to-compress mixture was pressed in tablets employing a pharmaceutical rotating compressor.

TABLE 4

| # | MATERIAL | Concentration | formulation % |
|---|---|---|---|
| 1 | LACTASE | Eq. a 9,000 FCC | 18.00 |
| 2 | MANNITOL | 200.00 mg | 40.00 |
| 3 | MICROCRYSTALLINE | 198.20 mg | 39.64 |
| | CELLULOSE | | |
| 4 | VANILLA TASTE | 1.80 mg | 0.36 |
| 5 | MICRONIZED | | |
| | STEARIC ACID | 10.00 mg | 2.00 |

### Example 3

In Example 3, lactase (9,000 FCC) was mixed with mannitol, L-HPC, vanilla taste, and croscarmellose, for 15 minutes in BIN-type mixer. Magnesium stearate was sieved, followed added, and mixed for 3 minutes. The ready-to-compress mixture was pressed in tablets employing a pharmaceutical rotating compressor.

TABLE 5

| # | MATERIAL | Concentration | formulation % |
|---|---|---|---|
| 1 | LACTASE | Eq. a 9,000 FCC | 18.00 |
| 2 | MANNITOL | 280.00 mg | 56.00 |
| 3 | L-HPC | 89.00 mg | 17.80 |
| 4 | VANILLA TASTE | 1.80 mg | 0.36 |
| 5 | CROSCARMELLOSE | 29.20 mg | 5.84 |
| 6 | MAGNESIUM STEARATE | 10 mg | 2.00 |

### Example 4

In Example 4, lactase (9,000 FCC) was mixed with mannitol, sylicated cellulose, sucralose, vanilla taste and crospovidone, for 15 minutes in “V” mixer. Magnesium stearate was sieved, followed added and mixed for 3 minutes. The ready-to-compress mixture was pressed in tablets employing a pharmaceutical rotating compressor.

TABLE 6

| # | MATERIAL | Concentration | formulation % |
|---|---|---|---|
| 1 | LACTASE | 9,000 FCC | 18.00 |
| 2 | MANNITOL | 180.00 mg | 36.00 |
| 3 | SYLICATED CELLULOSE | 198.16 mg | 39.63 |
| 4 | SUCRALOSE | 0.04 mg | 0.008 |
| 5 | VANILLA TASTE | 1.80 mg | 0.36 |
| 6 | CROSPOVIDONE | 20.00 mg | 4.00 |
| 7 | MAGNESIUM STEARATE | 10.00 mg | 2.00 |

### Example 5

Tablets obtained from previous examples are subjected to analysis related to process and quality control. As the object of said invention is fast disintegrating tablets, disintegrating time is a critical quality parameter; however, this parameter is closely linked to friability, hardness and average weight of tablets.

The method of each analysis applicable to orally disintegrating lactase tablets is described above.

### Friability:

Friability is performed in Friability Meter, apparatus consisting in rotating cylinder, rotating about its axis at a speed of 25 rotations per minute. The test establishes abrasion-resistance of tablets, when they are subjected to mechanic action and it is applied only to non-coated tablets. The test consisted in weighting exactly a certain number of tablets, submit them to apparatus function, and retrieve them after 100 rotations are completed. Twenty tablets were weighted and inserted in apparatus. The speed was adjusted to 25 rotations per minute and the apparatus time to 4 minutes. After this term, any powder residue was removed from tablets surface and they were weighted again. In accordance with Brazilian Pharmacopoeia (2010), none tablet may present any cracks, chip, rupture or breaks ate the end of test. Tablet with loss equal or lower than 1.5% of its weight are considered acceptable. If the result is doubtful or if its loss is higher than specified limit, test should be repeated for two times more, considering average result of three determinations on assessment. Friability test was performed in friability meter ERWEKA, model TAR120.

### Hardness:

Hardness test is applicable mainly to non-coated tablets, allowing to determine tablet resistance to crushing or rupturing under radial pressure, through apparatus called durometer. Analysis was performed as described in Brazilian Pharmacopoeia (2010), wherein 10 tablets were submitted, solely, to action of apparatus measuring diametrically applied force required for crushing it. The result is expressed as the average of values obtained in determinations. Hardness was determined using a durometer of brand ERWEKA, model TBH125.

### Average Weight:

Brazilian Pharmacopoeia reports that trial performed to establish average weight consists in individually weighting 20 tablets, in analytical scale, and divide total weight by amount of weighted units, obtaining average weight. To be in accordance with the acceptable parameters, average weight should not have more than two units out of specified limits, and no one may be above or under the indicated percentage, for 500 mg tablets the acceptable range is ±5.0%. The analysis was performed according to Brazilian Pharmacopoeia description (F. Bras. 5ª ed., 2010).

### Disintegration:

The disintegration test aims to submit six tablets to conditions similar those found in human body. It is performed through disintegrating apparatus that consist of a baskets and tubes system, container suited to immersion liquid (distilled water) and thermostat to maintain the liquid at 37° C. Samples were input and submitted to a moving system, ascending and descending, intending to produce the same effects which tablets are undergone following consumption and through the mouth, stomach, and bowel. Optimal time cutoff for disintegrating is 30 minutes for immediate-release tablets (F. Bras. 5ª ed., 2010), however, for orally disintegrating tablets is recommended the disintegrating time lower than 1.0 minute. Disintegrating test was performed in a disintegrator ERWEKA, model CT322.

### Results:

The obtained results average of analysis of orally disintegrating lactase tablets is shown below.

TABLE 7

| SAMPLES | RESULTS | SD | CV |
|---|---|---|---|
| Friability (Weight Loss - %) | 0.1% | | |
| Mean Hardness (Kgf) | 9.265 Kgf | 1.25 | 0.13 |
| Average weight (mg) | 501.91 mg | 8.93 | 0.018 |
| Disintegration (s) | 48″ | | |

Those well versed in the art will value the knowledge here, and may reproduce the invention in the manner provided and other variants, covered within the scope of appended claims.

1. A pharmaceutical composition for oral administration, wherein it is an orally disintegrating tablet comprising lactase enzyme in combination with pharmaceutically acceptable excipients.

2. The pharmaceutical composition, according to claim 1, wherein the pharmaceutically acceptable excipients comprises at least a disintegrating agent, a lubricant, a flavoring, a sweetener, and a diluent.

3. The pharmaceutical composition, according to claim 2, wherein the disintegrating agent is chosen from crospovidone, croscarmellose, glycated starch, polacrilin potassium, hydroxypropyl cellulose, microcrystalline cellulose, or combinations thereof.

4. The pharmaceutical composition, according to claim 2, wherein the lubricant is chosen from magnesium stearate, sodium stearyl fumarate, stearic acid, sodium citrate or combinations thereof.

5. The pharmaceutical composition, according to claim 2, wherein the diluent is chosen from several polyols (maltitol, mannitol and xylitol), microcrystalline cellulose, starch, talc, maltodextrin, sucrose, and dextrose or combinations thereof.

6. The pharmaceutical composition, according to claim 1, wherein it shows total disintegration in up to 1 minute and friability lower than 1.5%.

7. The pharmaceutical composition, according to claim 1, wherein it is mixed in lactose-rich foods.

8. A process to produce a pharmaceutical composition, according claim 1, wherein it comprises the steps of mixing of lactase with blend of diluents, disintegrating agents, and sweeteners, and next step of pressing.
